(19) United States
(12) Patent Application Publication
(10) Pub. No.: US 2004/0176649 A1
(43) Pub. Date: Sep. 9, 2004

(54) Making gas hydrate utilizing ultrafine bubbles and ultra-particulate gas hydrate

(75) Inventors: Masayoshi Takahashi, Tsukuba-shi (JP); Yoshitaka Yamamoto, Tsukuba-shi (JP)

Correspondence Address: SUGHRUE MION, PLLC, 2100 PENNSYLVANIA AVENUE, N.W., SUITE 800, WASHINGTON, DC, 20037, US

(73) Assignee: NATIONAL INSTITUTE OF ADVANCED INDUSTRIAL SCIENCE AND TECHNOLOGY

(21) Appl. No.: 10/790,716

(22) Filed: Mar. 3, 2004

(30) Foreign Application Priority Data
Mar. 4, 2003 (JP) 2003-057688

(51) Int. Cl. C07C 7/00; B01J 3/00

(52) U.S. Cl. 585/15; 422/242

(57) ABSTRACT

A method for making gas hydrate comprising generating ultrafine bubbles in an aqueous solution; and spontaneously generating hydrate nuclei by self-compression and collapsing of the ultrafine bubbles.

### DESCRIPTION

### RELATED APPLICATIONS

This Application claims foreign priority from JP 2003-057688, filed Mar. 4, 2003, the contents of which are incorporated herein by reference.

### FIELD

The present disclosure teaches techniques related to making gas hydrate.

### BACKGROUND

Related Work

A sufficiently large amount of gas must be dissolved into an aqueous solution at high pressure and low temperature for making gas hydrate. Processes for making gas hydrate can be classified into two main categories. In the first category, bubbles are generated by bubbling gas into an aqueous solution. In the second category, an aqueous solution is sprayed into the gas. In the former process dissolution efficiency can be improved by stirring the solution with propeller blades.

An apparatus for generating ultrafine bubbles by a swirling two-phase flow, and a method for making gas hydrate using this apparatus is described in Japanese Unexamined Patent Application Publication No. 2000-000447. The yield using this method is low. Therefore, a hydration accelerator is required to improve the yield. This is described further in Proceedings of the Fourth International conference on Gas Hydrate “A Novel Manufacturing Method of Gas Hydrate using the Micro-bubble Technology.”

Formation of nuclei is essential for generation of a solid phase gas hydrate in an aqueous solution. The generation of gas hydrate nuclei requires severe supercooling conditions. A large apparatus and a large amount of energy is required to achieve such severe supercooling conditions by merely adjusting the ambient pressure and temperature. Creating such severe supercooling conditions is known to be a technological challenge. Furthermore, it is difficult to effectively supply gas molecules that are required for growth of the hydrate after the hydrate nuclei have been formed. This causes a significant decrease in hydrate yield.

### SUMMARY

It will be significantly advantageous to overcome problems noted above in related art.

The disclosed teachings provide a method for making gas hydrate comprising generating ultrafine bubbles in an aqueous solution; and spontaneously generating hydrate nuclei by self-compression and collapsing of the ultrafine bubbles.

In a specific enhancement, a subset of the ultrafine bubbles have a diameter of 50 □m or less.

In another specific enhancement, a subset of the ultrafine bubbles exhibit an ascending rate of 1 mm/sec or less.

In another specific enhancement, the ultrafine bubbles are dissolved in the aqueous solution.

In yet another specific enhancement, the ultrafine bubbles are generated under a hydraulic pressure of more than 1 atm.

More specifically, the ultrafine bubbles are dissolved in the aqueous solution at a quantity larger than an amount of a corresponding gas that is normally dissolved at an ambient pressure.

In another specific enhancement, the gas hydrate nuclei are formed at a region of the solution above the metastable marginal curve by the collapsing phenomenon of the ultrafine bubbles.

In still another specific enhancement, the ultrafine bubbles are generated by a swirling two-phase flow process.

More specifically, the ultrafine bubbles are generated by a bell ultrafine-bubble generator.

Another aspect of the disclosed teachings is an apparatus for making a gas hydrate comprising an ultrafine bubble generator having an aqueous solution inlet, a gas inlet and an outlet for the aqueous solution containing ultrafine bubbles. The ultrafine bubble generator is placed in A high pressure vessel with aqueous solution. Ultrafine bubbles from the bubble generator ascend through the aqueous solution in the high pressure vessel. The hydrate nuclei are generated in the aqueous solution in the high pressure vessel by self-compression and collapsing of the ultrafine bubbles.

Still another aspect of the disclosed teachings is a articulate gas hydrate prepared by the techniques described herein.

### BRIEF DESCRIPTION OF THE DRAWINGS

The disclosed teachings will become more apparent by describing in detail examples and embodiments thereof with reference to the attached drawings in which:

FIG. 1 shows an example of an ultrafine-bubble generator.

FIG. 2 shows a schematic view of the ascending rate of ultrafine bubbles.

FIG. 3 shows a schematic view of shrinkage and collapse (observed value) of ultrafine bubbles.

FIG. 4 shows an example of an apparatus used for observing ultrafine bubbles and gas hydrate.

FIG. 5 shows a graph of the size distribution of ultrafine bubbles and gas hydrate particles.

FIG. 6 shows a schematic view of a mechanism for forming gas hydrate nuclei from ultrafine bubbles during the collapse stage.

### DETAILED DESCRIPTION

IV.A. Synopsis

The amount of gas dissolved in the vicinity of ultrafine bubbles in an aqueous solution is significantly increased by self-compression and collapsing of the ultrafine bubbles. This significantly increases the nucleation rate of the gas hydrate. Furthermore, the gas in the bubbles is effectively dissolved into the aqueous solution by the self-compression effect. The large specific area, and a long staying time of the ultrafine bubbles further contribute to the dissolution. This dissolved gas rapidly creates gas hydrate layers around the gas hydrate nuclei and gas hydrate that are preliminarily generated. As a result, gas hydrate is generated at a significantly improved rate.

Ultrafine bubbles with a diameter of 50 □m or less exhibiting an ascending rate of 1 mm/sec or less are generated in water. This is done under a hydraulic pressure of 1 atm or more in water to cause collapse of the bubbles. The collapse of the bubbles is due to self-compression of the ultrafine bubbles. Theoretically, an infinite increase in pressure occurs by the collapsing phenomenon. Therefore, a significantly high concentration of gas molecules are generated around the bubbles in the aqueous solution.

Since the condition shifts above the metastable marginal curve, the hydrate nuclei can be spontaneously generated. The ultrafine bubbles, which have a large specific area, have high solubility. Therefore, the ultrafine bubbles can supply gas molecules necessary for the growth of the hydrate.

IV.B. Examples Illustrating Concepts Underlying the Disclosed Teachings

Example of gases that can be used to generate gas hydrates using the disclosed teachings include hydrocarbons (such as methane, ethane, and propane), carbon dioxide and rare gases (such as argon, krypton, and xenon).

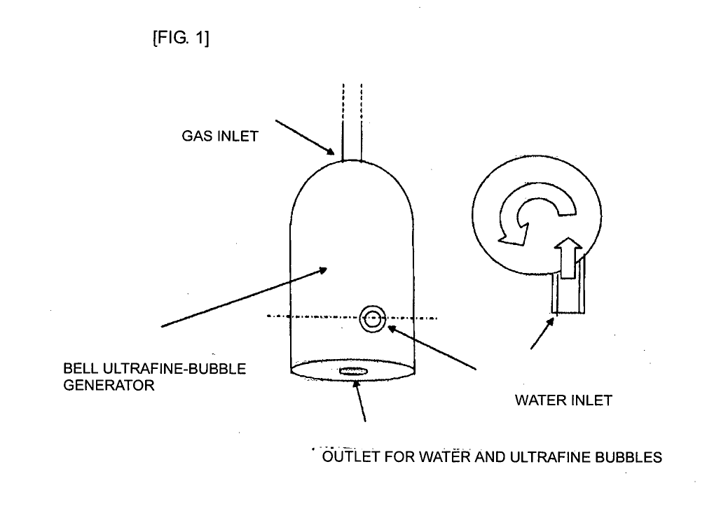

FIG. 1 shows a bell ultrafine-bubble generator 1 that generates ultrafine bubbles having a diameter of 50 μm or less. The hollow bell ultrafine-bubble generator 1 has a water inlet 2 and a gas inlet 3. An outlet 4 for water and ultrafine bubbles is provided. The hollow bell-generator placed in water. When water is supplied from the water inlet 2, while the ambient pressure and the water temperature are controlled, the water is circulated in the hollow bell. This circulation of water generates a centrifugal force that causes a reduction in pressure in the center of the bell. As a result, gas from the gas inlet 3 is drawn to the center to generate ultrafine bubbles.

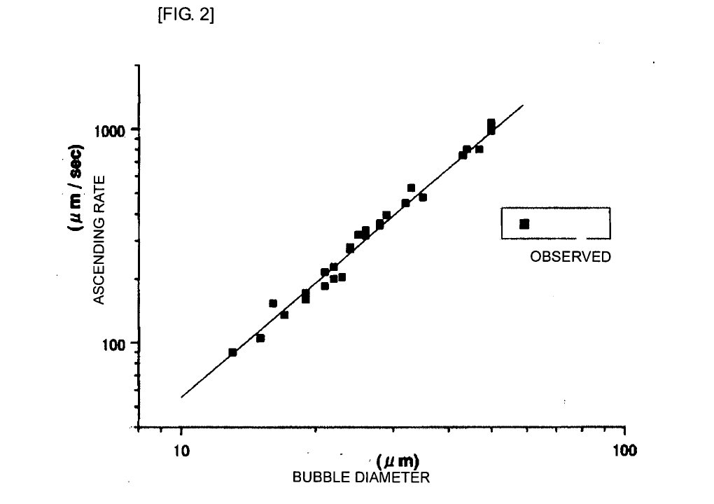

FIG. 2 illustrates an ascending rate of ultrafine bubbles. For example, bubbles having a diameter of 1 mm ascend at a rate of 100 mm/sec or more. Therefore, such bubbles ascending in water at a rate of 100 mm/sec instantaneously reaches the water surface and burst. However, an ascending rate of 1 mm or less, leads to a significantly long staying time. Because of this long staying time, these bubbles having an ascending rate below 1 ml/sec are dissolved into water and disappear therein.

Ultrafine bubbles having 50 μm or less have an ascending rate of 1 mm/sec or less in water at 1 atm or more. Moreover, these ultrafine bubbles exhibit a steep increase in internal pressure due to a self-compression effect and a collapsing phenomenon by surface tension. This behavior is not observed in larger bubbles.

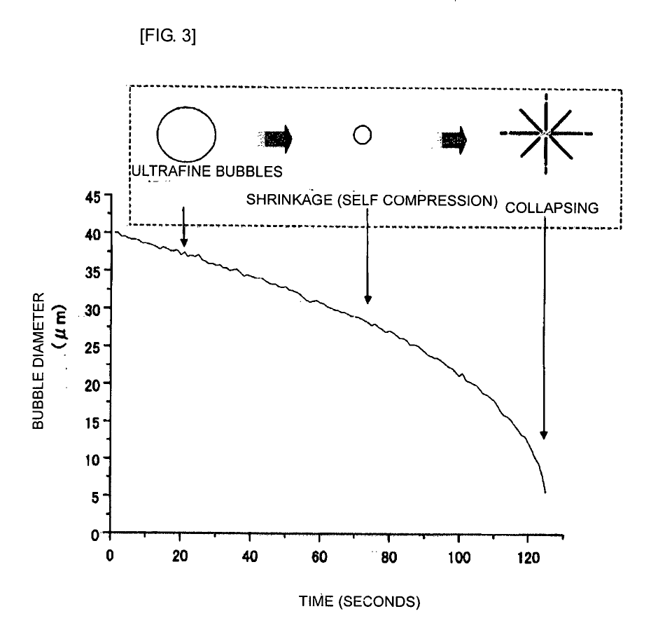

FIG. 3 illustrates the behavior of ultrafine bubbles from shrinkage to disappearance (collapse) in water. Although the time for disappearance varies with the ambient conditions such as temperature and pressure, such a behavior can be observed only in ultrafine bubbles having a diameter of 50 μm or less.

Herein, the internal pressure of the bubbles is represented by the following equation:

P_{g}=Pₗ+4S/d

wherein P_{g} indicates the internal pressure of the bubbles, Pₗ indicates the pressure of the aqueous solution (ambient pressure), S indicates the surface tension, and d indicates the diameter of bubbles.

At a collapsing stage (d=0) of shrunken bubbles, theoretically, the internal pressure becomes infinite.

According to calculations using distilled water, the pressure increases by 0.28 atm for bubbles with a diameter of 10 μm, 2.8 atm for a diameter of 1 μm, and 28 atm for a diameter of 0.1 μm. The time axis of the graph depends on the ambient conditions.

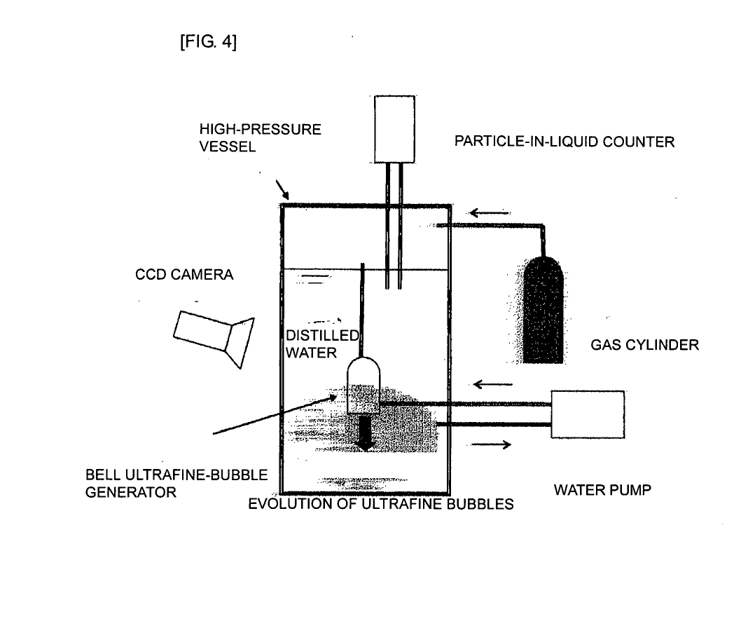

FIG. 4 shows an example of an apparatus for observing ultrafine bubbles and gas hydrate particles that are generated.

A high-pressure vessel 5 is provided with water. The bell ultrafine-bubble generator 1 is placed in the water. A water pump 6 and a gas cylinder 7 are operated to generate ultrafine bubbles in the water. A liquid particle counter 8 and a CCD camera 9 were equipped to observe the ultrafine bubbles generated.

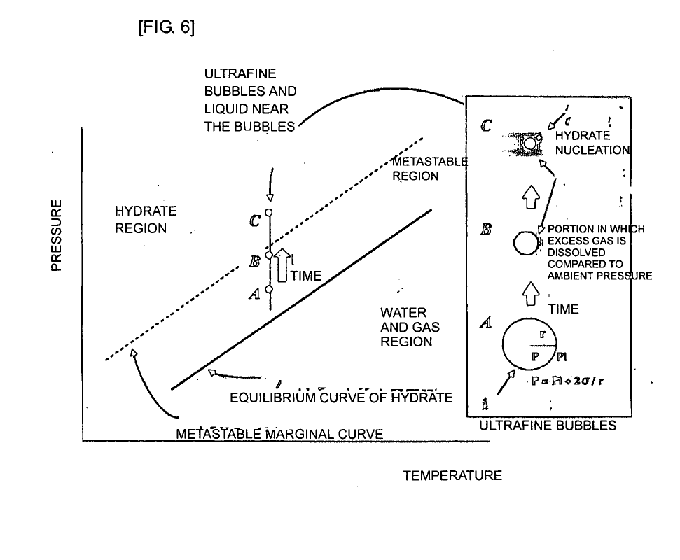

FIG. 6 illustrates a mechanism for generating gas hydrate nuclei from ultrafine bubbles in water.

As shown in FIG. 3, the ultrafine bubbles are shrunken and then are collapsed in the water. In this process, the internal pressure of the bubbles rapidly increases due to surface tension. At the collapsing stage (d=0), the internal pressure theoretically becomes infinite. A significantly high concentration of gas molecules is dissolved around ultrafine bubbles in proportion to the pressure of the bubbles.

Gas hydrate nuclei are spontaneously generated in the vicinity of the bubbles by this effect. In the metastable region shown in FIG. 6, the gas hydrate nucleation is a stochastic phenomenon. The probability of nucleation infinitely decreases near the equilibrium curve. In contrast, in a region above the metastable marginal curve, the hydrate nucleation occurs spontaneously and instantaneously.

In FIG. 6 point A represents an overall ambient condition. Conventionally, at this point, gas hydrate nuclei can be generated at a low probability. Using ultrafine bubbles, however, a high concentration of gas molecules is dissolved around the bubbles during the self shrinking stage. Point B represents a stage in which a excess gas is dissolved as compared to the dissolution at ambient pressure. Point C represents a stage at which nucleation is begun. The condition of the aqueous solution varies from point A to point B and then to point C in the vicinity of these bubbles.

Since the pressure is expected to increase to infinite, the condition shifts above the metastable marginal curve. As a result, even at point A, spontaneous gas hydrate nucleation is achieved. Since point A functions as a stable region for gas hydrate, the nuclei generated spontaneously grow to gas hydrate particles.

The continuously generated ultrafine bubbles also play a role of supplying gas molecules, necessary for growth of hydrate, to the aqueous solution. The ultrafine bubbles, which have a large specific area, have high solubility in the solution. During generation of hydrate, bubble bursting is not observed at the water surface. This suggests that bubbles are effectively collapsed and bubbles effectively supply gas molecules necessary for hydrate growth.

The following examples illustrate some implementations of the disclosed teachings.

IV.C. Example I

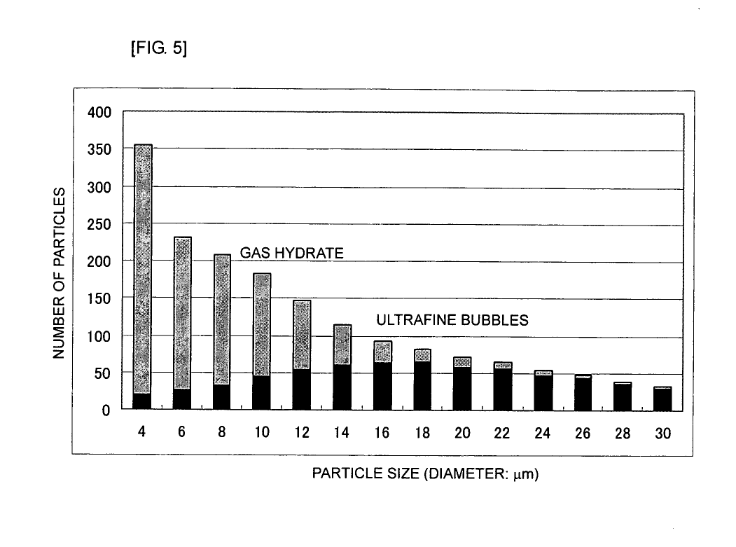

In a high-pressure vessel, xenon (Xe) ultrafine bubbles were released in distilled water to study the conditions for generating hydrate. A swirling two-phase flow system was utilized for making ultrafine bubbles. The pressure was 0.3 MPa (gauge pressure) and the water temperature was 8.0° C. The ultrafine-bubble generator was operated for 3 minutes. The particle size distribution of the ultrafine bubbles at that time is shown by a filled bar graph in FIG. 5.

One minute after the shutoff of the generator, generation of gas hydrate particles was observed. The particle size distribution of the ultrafine bubbles at about three minutes from the shutoff is shown by a grey bar graph in FIG. 5. The graph indicates that the number of hydrate particles is significantly greater than the bubble distribution. This shows that the accumulated gas hydrate nuclei grow with the elapsed time to a size that can be measured with a particle-in-liquid counter. The fact that finer particles are abundant shows growth of hydrate particles at the time of the measurement.

The water temperature was increased under the same pressure condition, and disappearance of the hydrate was observed at 8.7° C. This shows that the equilibrium condition of the gas hydrate is about 8.7° C. For the same pressure, supercooling of at least 4° C. from the equilibrium condition is required for a conventional method. On the other hand, supercooling of merely 0.7° C. from the equilibrium condition could generate gas hydrate according to the method utilizing disclosed technique involving ultrafine bubbles.

According to the disclosed teachings, gas hydrate nuclei can be spontaneously generated by a self-compression effect and a collapsing phenomenon of ultrafine bubbles having a diameter of 50 μm or less and an ascending rate of 1 mm/sec or more. As a result, gas hydrate can be effectively generated at a significant rate.

Due to the effect of the large specific area of the ultrafine bubbles gas molecules required for a significant growth of gas hydrate in an aqueous solution can be effectively supplied.

Other modifications and variations to the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention.

What is claimed is:

1. A method for making gas hydrate comprising:
a) generating ultrafine bubbles in an aqueous solution; and
b) spontaneously generating hydrate nuclei by self-compression and collapsing of the ultrafine bubbles.

2. The method of claim 1, wherein a subset of the ultrafine bubbles have a diameter of 50 μm or less.

3. The method of claim 1, wherein a subset of the ultrafine bubbles exhibit an ascending rate of 1 mm/sec or less.

4. The method of claim 1, wherein the ultrafine bubbles are dissolved in the aqueous solution.

5. The method of claim 1, wherein the ultrafine bubbles are generated under a hydraulic pressure of more than 1 atm.

6. The method of claim 4, wherein the ultrafine bubbles are dissolved in the aqueous solution at a quantity larger than an amount of a corresponding gas that is normally dissolved at an ambient pressure.

7. The method of claim 1, wherein the gas hydrate nuclei are formed at a region of the solution above the metastable marginal curve by the collapsing phenomenon of the ultrafine bubbles.

8. The method of claim 1, wherein the ultrafine bubbles are generated by a swirling two-phase flow process.

9. The method of claim 8, wherein the ultrafine bubbles are generated by a bell ultrafine-bubble generator.

10. An apparatus for making a gas hydrate comprising:
an ultrafine bubble generator having an aqueous solution inlet, a gas inlet and an outlet for the aqueous solution containing ultrafine bubbles;
a high pressure vessel with aqueous solution having the ultrafine bubble generator place therein; and
ultrafine bubbles from the bubble generator ascending through the aqueous solution in the high pressure vessel,
wherein hydrate nuclei are generated in the aqueous solution in the high pressure vessel by self-compression and collapsing of the ultrafine bubbles.

11. The apparatus of claim 10, wherein a subset of the ultrafine bubbles have a diameter of 50 μm or less.

12. The apparatus of claim 10, wherein a subset of the ultrafine bubbles exhibit an ascending rate of 1 mm/sec or less.

13. The apparatus of claim 10, wherein the ultrafine bubbles are dissolved in the aquesous solution.

14. The apparatus of claim 10, wherein the ultrafine bubbles are generated under a hydraulic pressure of more than 1 atm.

15. The method of claim 13, wherein the ultrafine bubbles are dissolved in the aqueous solution at a quantity larger than an amount of a corresponding gas that is normally dissolved in an ambient pressure.

16. The method of claim 10, wherein the gas hydrate nuclei are formed at a region of the solution above the metastable marginal curve by the collapsing phenomenon of the ultrafine bubbles.

17. The method of claim 10, wherein the ultrafine bubbles are generated by a swirling two-phase flow process.

18. Particulate gas hydrate prepared by the method for making gas hydrate according to a following process:
a) generating ultrafine bubbles in an aqueous solution; and
b) spontaneously generating hydrate nuclei by self-compression and collapsing of the ultrafine bubbles.
